(19) United States
(12) Patent Application Publication
(10) Pub. No.: US 2016/0113817 A1
(43) Pub. Date: Apr. 28, 2016

(54) SLEEPING AID MASK

(71) Applicant: Jan-Mao Chen, New Taipei City (TW)

(72) Inventor: Jan-Mao Chen, New Taipei City (TW)

(21) Appl. No.: 14/520,587

(22) Filed: Oct. 22, 2014

(51) Int. Cl. A61F 9/04; A61M 21/02

(52) U.S. Cl. CPC A61F 9/04; A61M 21/02; A61M 2021/0016

(57) ABSTRACT

A sleeping aid mask, comprising a mask, a fixing element, and a porous sack. Wherein, said mask body is formed by light shielding cloth; said fixing element, formed at both ends of the mask body; and said porous sack is disposed at a lower portion of the mask corresponding to user's nose, inside said porous sack is placed a water absorbing material, and a sleeping aid oil is disposed in that material, so that fragrance of said sleeping aid oil penetrates through holes of said porous sack, and then enters into user's nose. As such, in application, said mask body is placed over user's eyes and nose, and fragrance of said sleeping aid oil dissipated through said porous sack is entered into user's nose, to actively help the user to relax, to relieve user of tension, thus helping the user fall into sleep.

### BACKGROUND OF THE INVENTION

1. Field of the Invention

The present invention relates to a mask, and in particular to a sleeping aid mask, that utilizes fragrance of sleeping aid oil to help the user fall into sleep.

2. The Prior Arts

Nowadays, with the progress and development of business and industry, the work tempo and life style have changed drastically. The quick tempo life style and busy work schedule usually make people feel nervous and unrest, and not easy to get into sleep. As such, not only the time of our sleep is decreased, but the quality of our sleep is also reduced. In recent years, this type of sleeping obstacle is getting ever more serious, so that people can not get adequate and enough rest and sleep as required. The phenomenon could make people getting drowsy, nervous, and irritable, hereby leading to various chronic diseases.

In order to prevent people from addicting to drug (such as sleeping pills) to fall into and to have enough sleep, a kind of eye mask is developed to help people to fall into sleep easily. In this approach, the eye mask is used to cover user's eyes during sleep, to shield off the excessive lights, and to protect the eyes of the users from exposure to too much lights, such that the user feels relaxed and relieved of pressure and tension, to fall into a sound and nice sleep.

However, for the eye mask presently available on the market, that is capable of only shielding lights from getting to the eyes, to help people fall into sleep in a passive way. Yet, for the people having difficulty falling into sleep, this type of eye mask is not able to actively make user feel relaxed, relieved of his tensions, and fall easily into sleep.

Therefore, presently, the design and performance of eye mask is not quite satisfactory, and it has much room for improvement.

### SUMMARY OF THE INVENTION

In view of the problems and drawbacks of the prior art, the present invention provides a sleeping aid mask, to effectively overcome the shortcomings of the prior art.

A major objective of the present invention is to provide a sleeping aid mask, that utilizes the fragrance of the sleeping aid oil to help the user fall into sleep, to actively make the user feel relaxed, relieved of his tensions, in realizing a nice and sound sleep.

In order to achieve the objective mentioned above, the present invention provides a sleeping aid mask, mainly includes: a mask body, a porous sack, and a fixing element. Wherein, the mask body is formed by light shielding cloth; the fixing element is formed at both ends of the mask body, so that when the fixing element is fixed around and behind the back of user's head, the mask is covered over the eyes and nose of a user. The porous sack is disposed at the lower portion of the mask corresponding to the nose of the user. Inside the porous sack is placed a replaceable water absorbing material, and a sleeping aid oil is absorbed in that material, so that the fragrance of the sleeping aid oil can penetrate through the holes of the porous sack, and then it enters into the nose of the user.

In application, the mask is put over the eyes of the user, so that the fragrance of the sleeping aid oil is dissipated through the porous sack, and enters into user's nose, to actively help the user feel relaxed, relieve user's tension, in helping the user to fall easily into sleep.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from this detailed descriptions.

### BRIEF DESCRIPTION OF THE DRAWINGS

The related drawings in connection with the detailed descriptions of the present invention to be made later are described briefly as follows, in which:

FIG. 1 is a schematic diagram of a sleeping aid mask according to an embodiment of the present invention; and

FIG. 2 is a schematic diagram of a sleeping aid mask according to another embodiment of the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings.

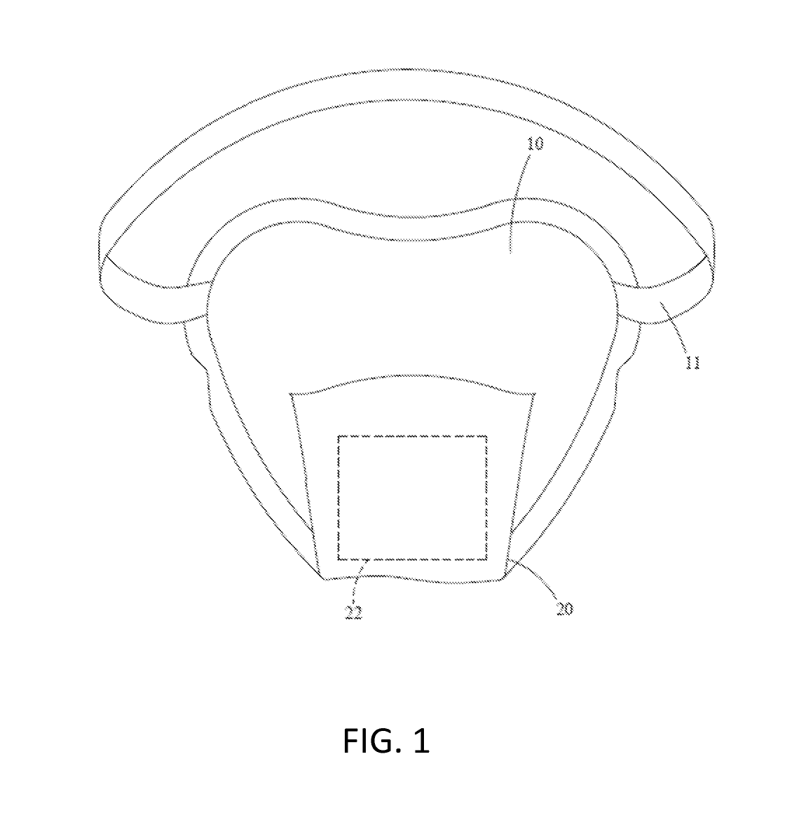

Refer to FIG. 1 for a schematic diagram of a sleeping aid mask according to an embodiment of the present invention. As shown in FIG. 1, the sleeping aid mask, includes a mask body 10, a fixing element 11, and a porous sack 20.

Wherein, the mask body 10 is formed by light shielding cloth.

The fixing element 11 is formed at both ends of the mask body 10, so that when the fixing element 11 is fixed around and behind the back of user's head, the mask body 10 is covered over the eyes and nose of a user, as such shielding lights from getting into the eyes of the user, to help him fall into sleep.

The porous sack 20 is disposed in the lower portion of the mask body 10 corresponding to user's nose, Inside the porous sack 20 is placed a water absorbing material 22, and a sleeping aid oil (not shown) is disposed in that material, so that the fragrance of the sleeping aid oil can penetrate through the holes (not shown) of the porous sack, and then enters into the nose of the user.

In application, the mask body 10 is put over the eyes and nose of the user, so that the fragrance of the sleeping aid oil is dissipated through the holes of the porous sack 20, and enters into the nose of the user, to actively make the user feel relaxed, and to relieve user of tension, in helping the user fall easily into sleep, without having to rely on the sleeping pills.

Refer to FIG. 1 for a schematic diagram of a sleeping aid mask according to an embodiment of the present invention. As shown in FIG. 1, the fixing element 11 can be an elastic rubber band.

Also, as shown in FIG. 1, the sleeping aid oil can be selected from one of a group consisting of: rose essence oil, rosemary essential oil, geranium essential oil, teaseed oil, cypress essential oil, terebinth oil, frankincense essential oil, mugwort oil, and lavender essential oil.

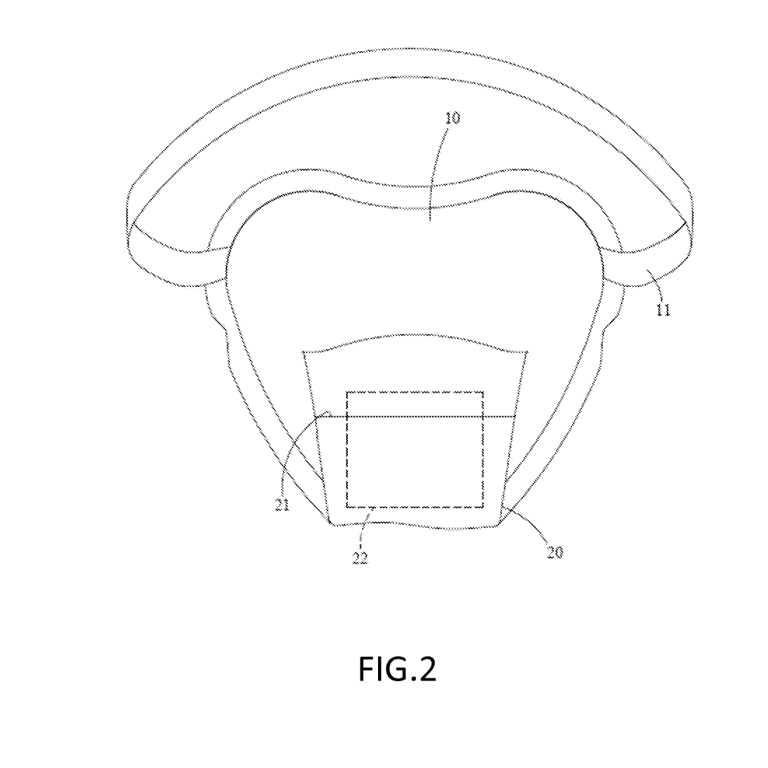

Then, refer to FIG. 2 for a schematic diagram of a sleeping aid mask according to another embodiment of the present invention. As shown in FIG. 2, the porous sack 20 is provided with a sack opening 21, such that the water absorbing material 22 can be put into the porous sack 20 through the sack opening 21. Also, water absorbing material 22 can be taken out from the porous sack 20 and be replaced through the sack opening 21. As such, through replacing the water absorbing material 22, the sleeping aid oil can be replaced.

Summing up the above, the sleeping aid mask of the present invention is of a novel design, that has not been published in any publication and is not available on the market. As such, it does have novelty. In addition, the functions and effectiveness of the present invention is not found in the prior art, such that it does fulfill the requirement of Non-obviousness.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A sleeping aid mask, comprising:
a mask body, formed by light shielding cloth,;
a fixing element, formed at both ends of said mask body, so that when said fixing element is fixed around and behind the back of user's head, said mask body is covered over eyes and nose of a user, to shield lights from getting into eyes of user, to help him fall into sleep; and
a porous sack, disposed at a lower portion of said mask corresponding to user's nose, inside said porous sack is placed a water absorbing material, and a sleeping aid oil is disposed in that material, so that fragrance of said sleeping aid oil penetrates through holes of said porous sack, and then enters into user's nose, such that in application, said mask is placed over user's eye and nose, and fragrance of said sleeping aid oil dissipated through said porous sack is entered into user's nose, to actively help the user to feel relaxed, to relieve user of tension, thus helping the user fall into sleep.

2. The sleeping aid mask as claimed in claim 1, wherein said fixing element is an elastic rubber band.

3. The sleeping aid mask as claimed in claim 1, wherein said sleeping aid oil is selected from one of a group consisting of: rose essence oil, rosemary essential oil, geranium essential oil, teaseed oil, cypress essential oil, terebinth oil, frankincense essential oil, mugwort oil, and lavender essential oil.

4. The sleeping aid mask as claimed in claim 1, wherein said porous sack is provided with a sack opening, such that said water absorbing material is put into said porous sack through said sack opening; and said water absorbing material is taken out from said porous sack and be replaced through said sack opening.
